(19) United States
(12) Patent Application Publication
(10) Pub. No.: US 2022/0397508 A1
(43) Pub. Date: Dec. 15, 2022

(54) ELECTRIC METER FOR MEASURING METAL IMPURITIES IN FUEL TANK

(71) Applicant: CHIAN -YEU CHIEN, New Taipei City (TW)

(72) Inventor: CHIAN -YEU CHIEN, New Taipei City (TW)

(21) Appl. No.: 17/364,911

(22) Filed: Jul. 1, 2021

(30) Foreign Application Priority Data
Jun. 11, 2021 (TW) 110121530

(51) Int. Cl. G01N 15/06; G01N 33/28

(52) U.S. Cl. CPC G01N 15/0656; G01N 33/2835; G01N 2015/0687

(57) ABSTRACT

An electric meter for measuring metal impurities in a fuel tank, comprising: a frame; two electric electrodes supported by the frame; an electric measurement meter connected to the two electric electrodes by using two conductive wires; the electric measurement meter serving to measure the electric properties between the two electric electrodes. In use, the frame with the two electric electrodes is placed into a fuel tank and is sunk into the bottom of the fuel tank; and the electric force of the two electric electrodes will attract the metal impurities in the fuel tank so that the electric measurement meter can measure the electric properties between the two electric electrodes; by the electric property, it can be used to determine the quantity of the metal impurities within the fuel tank so as to determine whether the fuel tank exists too much metal impurities and is needed to be cleaned.

### FIELD OF THE INVENTION

The present invention is related to cleaning of an fuel tank, and in particular to an electric meter for measuring metal impurities in a fuel tank.

### BACKGROUND OF THE INVENTION

In manufacturing, transportation and storage of gasoline, gasoline must contact with different kinds of manufacturing tools, devices and storage tanks, generally, these tools, devices or tanks contain large amount of metals which will generate with many metal impurities such as iron powders, rust powders, or impurities of metal particles or powders. These impurities will mix into the gasoline and then flow into the oil tanks of vehicles and sink in the bottom of the oil tank. For a long time, these impurities will seal the oil pump to cause that the pump cannot act and thus gasoline will not flow out of the oil tank to provide to the engine.

Above mentioned conditions will cause the vehicles cannot move so as to induce traffic jams abruptly and thus cause the traffic chaos, as a result, the driver needs to hire a tow truck to move the destroyed vehicle away. All these actions are time and money wasted.

In general maintenance works, the worker does not check whether the interior of the oil tank is dirty and how much metal powders stayed therein. This is because these works must be performed with the work of opening the oil tank, while to open the oil tank is a very difficult work.

Therefore, the object of the present invention is to provide a novel structure which can check whether the oil tank is needed to be cleaned so as to avoid the destroy of the oil tank due to the seal of the oil pump.

### SUMMARY OF THE INVENTION

Accordingly, the object of the present invention is to provide an electric meter for measuring metal impurities in a fuel tank, wherein the method to determine whether a fuel tank is clean is easy. The user is only necessary to check the value displayed on the electric measurement meter and then the user can determine the volume of the metal impurities sunk in the fuel tank. Therefore, the driver of a vehicle is very convenient and it is an easy way to prevent the oil pump in the fuel tank from non-operation. Therefore, the vehicle driving in a road can prevent from destroy due to the non-operation of the oil pump.

To achieve above object, the present invention provides an electric meter for measuring metal impurities in a fuel tank, comprising: a frame; two electric electrodes supported by the frame; the electric polarities of the two electric electrodes being opposite, electric field lines emitting from one electric electrode to another electric electrode; an electric measurement meter connected to the two electric electrodes by using two conductive wires; one end of each conductive wire fixed to a respective electric electrode, and the other end of the conductive wire being connected to the electric measurement meter; the electric measurement meter serving to measure the electric properties between the two electric electrodes; wherein the electric property is mainly affected by the metal impurities between the two electric electrodes; the electric properties are such as resistance, reactance, capacitance, inductance, voltage, current, frequency, etc., the metal impurities is such as iron powders or particles, or powders or particles of other metal, etc. within a fuel tank; and wherein in use, the frame with the two electric electrodes is placed into a fuel tank and is sunk into the bottom of the fuel tank; and the electric force of the two electric electrodes will attract the metal impurities in the fuel tank and the metal impurities are distributed along the electric lines so that the electric measurement meter can measure the electric properties between the two electric electrodes; by the electric property, it can be used to determine the quantity of the metal impurities within the fuel tank so as to determine whether the fuel tank exists too much metal impurities and is needed to be cleaned.

### BRIEF DESCRIPTION OF THE DRAWINGS

FIG. 1 is an assembly schematic view of the present invention.

FIG. 2 is a schematic view showing the first arrangement in the first embodiment of the present invention.

FIG. 3 is a schematic view showing the another arrangement in the first embodiment of the present invention.

FIG. 4 is a schematic view showing the distribution of the magnetic line according to the present invention.

FIG. 5 is an assembly schematic view about the arrangement showing in FIG. 2.

FIG. 6 is an exploded view of the second embodiment of the present invention.

FIG. 7 is an assembly view of the second embodiment of the present invention.

FIG. 8 shows an application of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

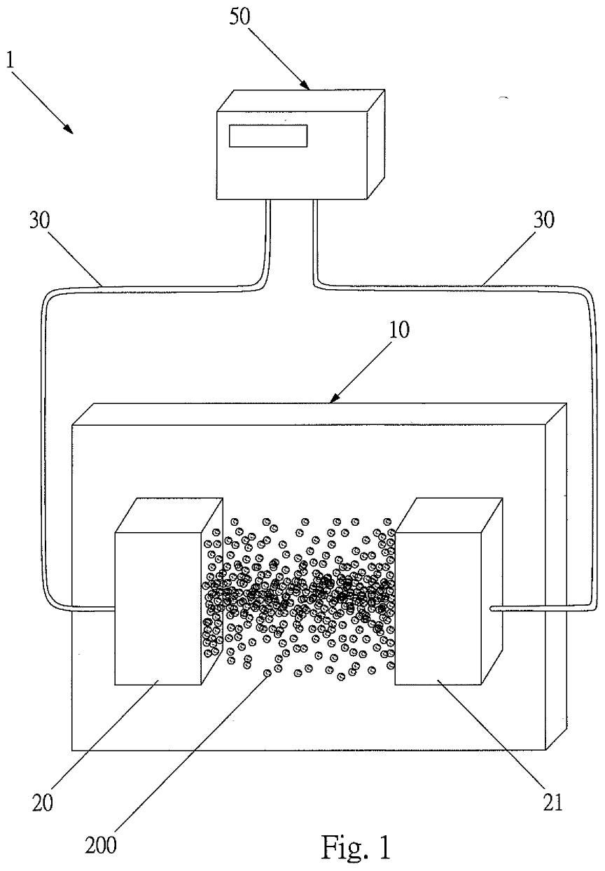

With reference to FIGS. 1 to 8, the structure according to the present invention is illustrated. The present invention includes the following elements.

A frame 10 is illustrated in FIG. 1. In the drawing, the frame 10 is a back plate, but this is not used to confine the scope of the present invention.

Two electric electrodes 20, 21 are supported by the frame 10. The electric polarities of the two electric electrodes 20, 21 are opposite, one is positive and the other is negative. Electric field lines emits from one electric electrode 20 to another electric electrode 21. Metal impurities are distributed along the electric field lines.

An electric measurement meter 50 is connected to the two electric electrodes 20, 21 by using two conductive wires 30. One end of each conductive wire 30 is fixed to a respective electric electrode, and the other end of the conductive wire 30 is connected to the electric measurement meter 50. The electric measurement meter 50 serves to measure the electric properties between the two electric electrodes 20, 21. In the present invention, the electric property is mainly affected by the metal impurities 200 between the two electric electrodes 20, 21. The electric properties are such as resistance, reactance, capacitance, inductance, voltage, current, frequency, etc. In the present invention, the metal impurities 200 is such as iron powders or particles, or powders or particles of other metal, etc. within a fuel tank 100.

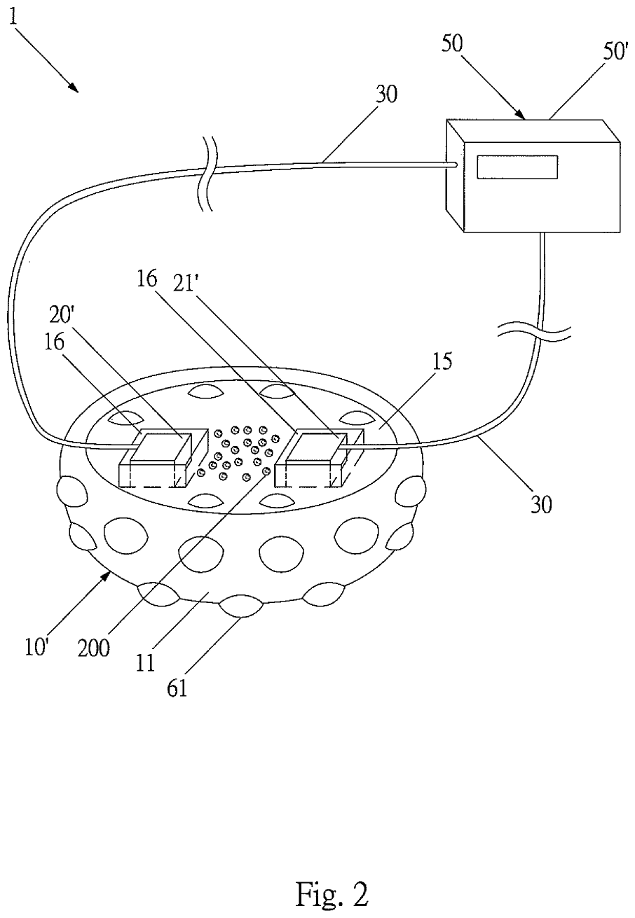

The first embodiment of the present invention is shown in FIGS. 2, 3, 4 and 5. In this embodiment, the frame 10 is a fuel tank measurement ball 10 which is non-magnetic and the magnetic lines can pass therethrough. Material of the measurement ball 10′ is such as rubber, plastic, etc. Referring to FIG. 2, an outer surface of the measurement ball 10′ is concave so as to have a larger area for attracting metal impurities 200, such as iron powders or particles, or powders or particles of other metal, etc.

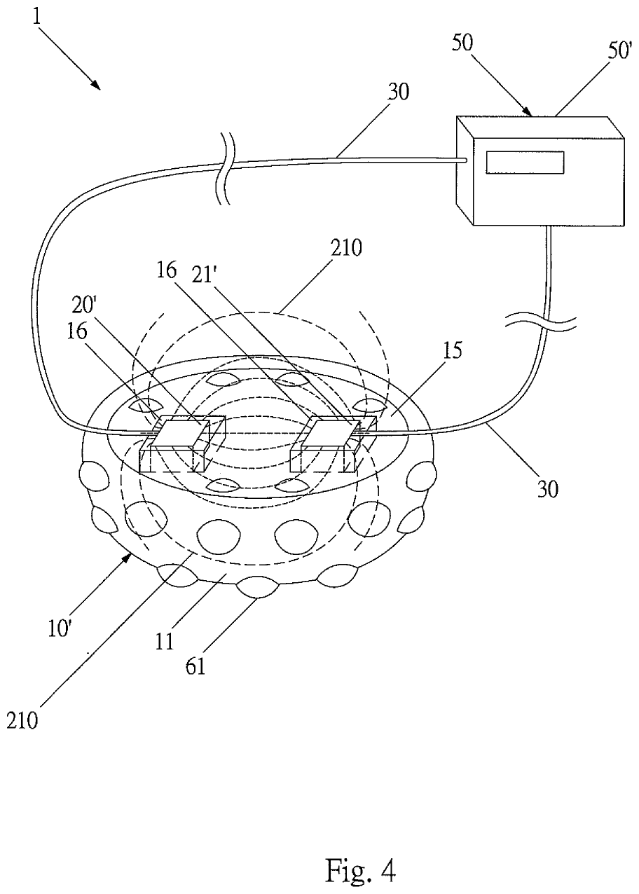

In this embodiment, the two electric electrodes 20, 21 are also two magnetic electrodes 20′, 21′ which are located at a platform surface 15 of the measurement ball 10′. The polarities of the two magnetic electrodes 20′, 21′ are opposite. With reference to FIG. 4, magnetic lines emitting from one magnetic electrode 20′ to another magnetic electrode 21′ so that magnetic material will distribute along the magnetic lines.

FIGS. 2, 4 and 5 shows that in the first embodiment, the first form of the two magnetic electrodes 20′, 21′, in that, the platform surface 15 are formed with two recesses 16 for locating the two magnetic electrodes 20′, 21′.

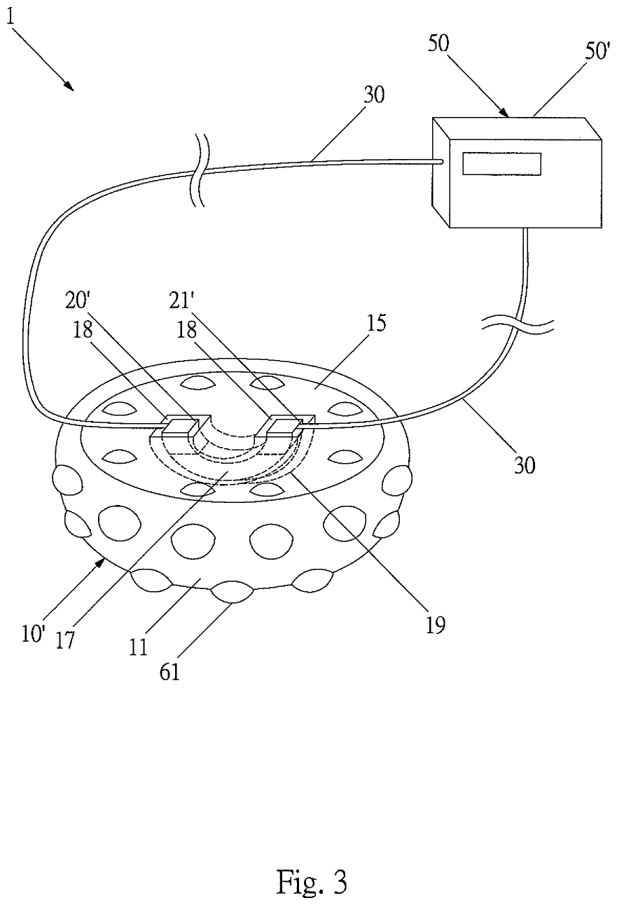

FIG. 3 shows another form of the two magnetic electrodes 20′, 21′, in that, an opened channel 19 is formed in the measurement ball 10′ with two openings 18 which are in the platform surface 15. A non-magnetic bar 17 with two magnetic electrodes 20′, 21′ at two ends thereof is received in the channel 19 with the two magnetic electrodes 20′, 21′ being exposed in the two openings 18.

Preferably, the magnetic force between the two magnetic electrodes 20′, 21′ is properly adjusted so that the magnetic force is formed only in the nearby of the measurement ball 10′. Thereby, frame 10 with the two electric electrodes 20, 21 placed within the fuel tank 100 is not adhered to a wall of the fuel tank 100.

Because the material of the measurement ball 10 is non-magnetic and is capable to be passed through by the magnetic lines, metal impurities 200 within the fuel tank 100 can be attracted by the measurement ball 10′.

In this embodiment, the electric measurement meter 50 is a meter for measuring reactance and resistance.

In this embodiment, one end of each of the conductive wires 30 is connected to a respective one of the two magnetic electrodes 20′, 21′ and another end thereof is connected to one end of the electric measurement meter 50 so as to measure the resistance between the two magnetic electrodes 20′, 21′.

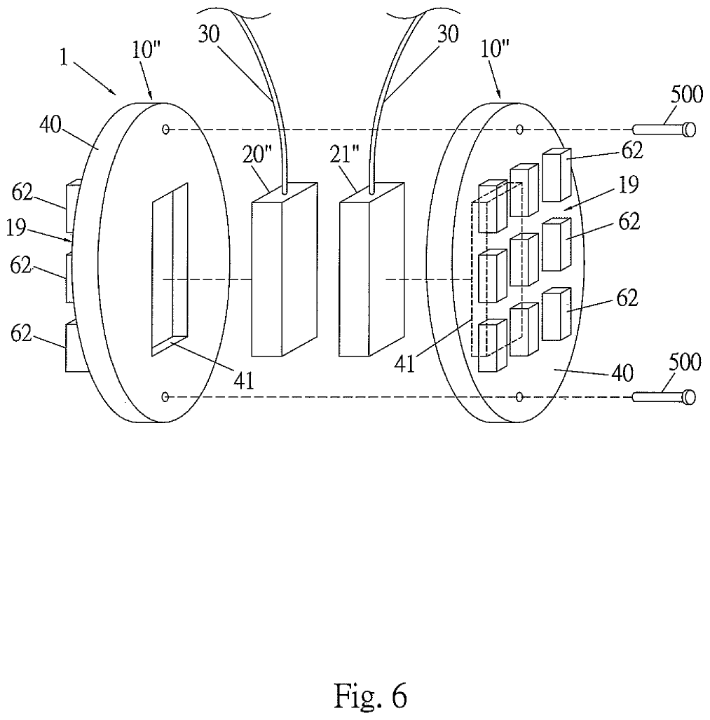

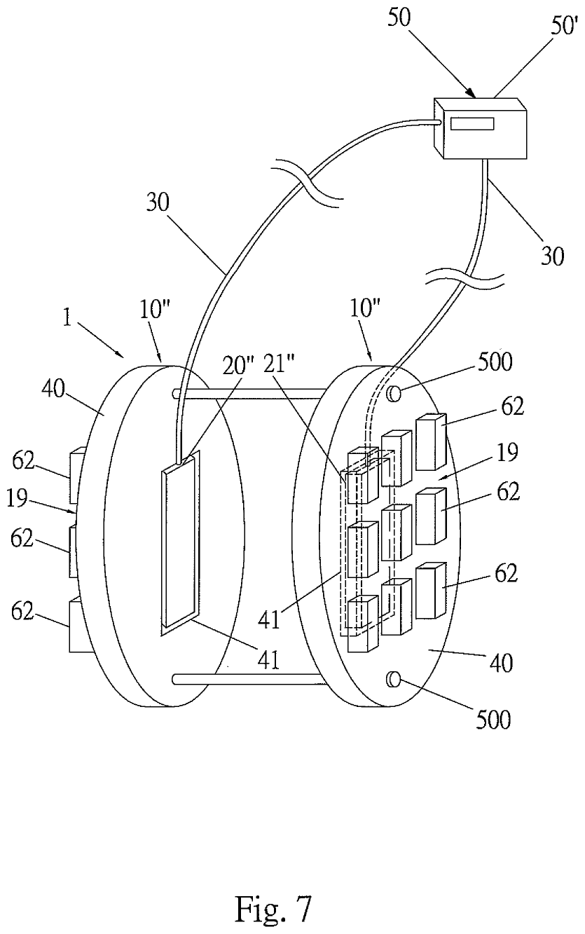

The second embodiment of embodiment will be described herein with reference to FIGS. 6 and 7, herein the frame 10 is an enclosure 10″ which is non-magnetic and the magnetic lines can pass therethrough. Material of the enclosure 10″ is such as rubber, plastic, etc. An outer surface of the enclosure 10″ is concave so as to have a larger area for attracting metal impurities 200.

In this embodiment, the two electric electrodes 20, 21 are also two magnetic electrodes 20″, 21″ which are enclosed and fixed within the enclosure 10″. Preferably, the magnetic force of the two magnetic electrodes 20″, 21″ are adjusted so that the magnetic force is only existed in the nearby of the enclosure 10″ so that when in the enclosure 10″ is placed into a fuel tank 100, it is not attracted to the wall of the fuel tank 100.

In this embodiment, one end of each of the conductive wires 30 is connected to a respective one of the two magnetic electrodes 20″, 21″ and another end further is connected to one end of the electric measurement meter 50 so as to measure the electric properties (such as resistance, reactance, capacitance or frequency) between the two magnetic electrodes 20′, 21′.

In this embodiment, the enclosure 10″ is two sheet bodies 40. Each sheet body 40 has a recess 41. In assembly, the two sheet bodies 40 are combined together by any prior art, such as screwing, using stud, gluing, buckling, etc. With reference to FIGS. 6 and 7, it is illustrated that two screws 500 are used to combine the two sheet bodies 40. However, the material to combine the sheet bodies 40 must not be conductive so as to avoid the two sheet bodies 40 from conduction to each other. The two magnetic electrodes 20″, 20″ are placed in the respective recesses 41. Because the two magnetic electrodes 20″, 20″ are made of non-magnetic material and magnetic lines can passed therethrough so that magnetic lines of the two magnetic electrodes 20″, 20″ can pass through the sheet bodies 40 to attract the metal impurities 200. At this embodiment, the two sheet bodies 40 with the two magnetic electrodes 20″, 20″ are acted as a capacitor.

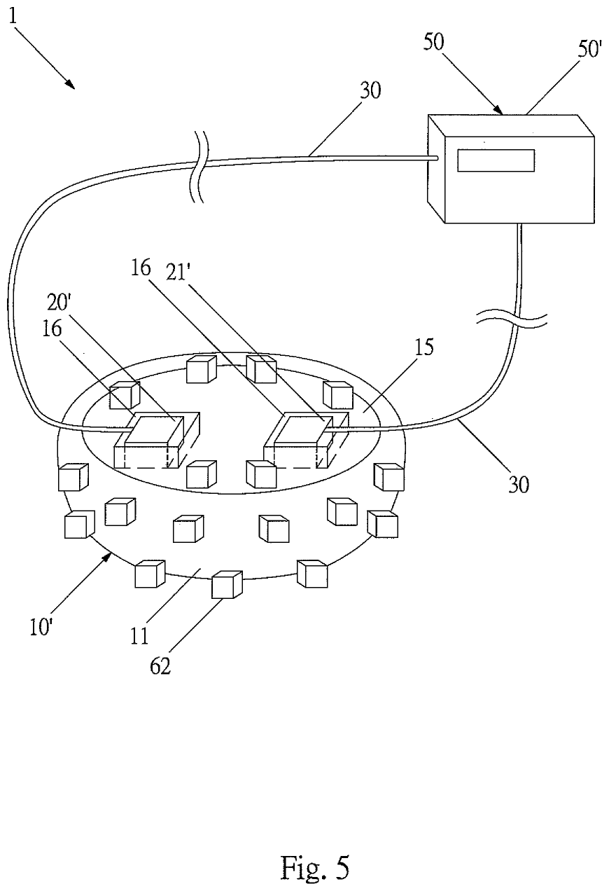

In the present invention, the outer side of the measurement ball is non-flat or concave so as to provide a larger surface for attracting metal impurities 200. In the present invention, the outer side of the measurement ball may be formed with a plurality of round posts 61 (see FIG. 4) or a plurality of rectangular posts 62 (see FIG. 5)

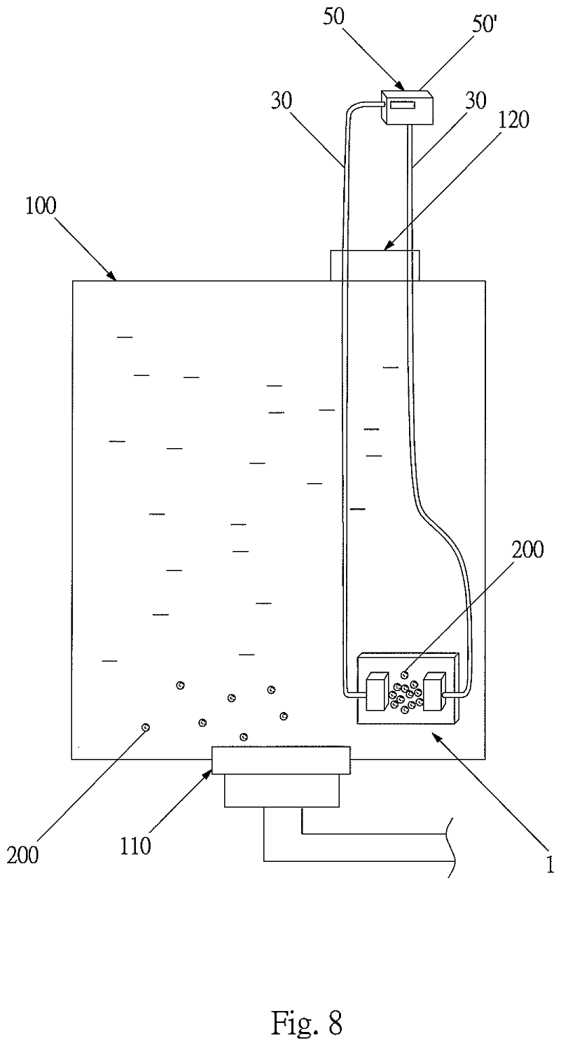

Referring to FIG. 8, in use, the frame 10 with the two electric electrodes 20, 21 is placed into a fuel tank 100 and is sunk into the bottom of the fuel tank 100. The electric force of the two electric electrodes 20, 21 (or magnetic force from the two magnetic electrodes 20′, 21′) will attract the metal impurities 200 in the fuel tank 100 and the metal impurities 200 are distributed along the electric lines so that the electric measurement meter 50 can measure the electric properties between the two electric electrodes 20, 21. By the electric property, it can be used to determine the quantity of the metal impurities 200 within the fuel tank. For example, if too much metal impurities 200 are distributed between the two electric electrodes 20, 21, the electric resistance will reduce between the two electric electrodes 20, 21.

Generally, these metal impurities 200 in the fuel tank will flow to an oil pump 110 in the fuel tank 100 so as to block the pump 110 so that the pump 110 cannot operate. In use, the electric measurement meter 50 is exposed out of an opening 120 of the fuel tank 100 so that the user can view the measured value and determine whether the fuel tank is necessary to be cleaned.

Advantages of the present invention are that the method to determine whether a fuel tank is clean is easy. The user is only necessary to check the value displayed on the electric measurement meter and then the user can determine the volume of the metal impurities sunk in the fuel tank. Therefore, the driver of a vehicle is very convenient and it is an easy way to prevent the oil pump in the fuel tank from non-operation. Therefore, the vehicle driving in a road can prevent from destroy due to the non-operation of the oil pump.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An electric meter for measuring metal impurities in a fuel tank, comprising:
a frame;
two electric electrodes supported by the frame; the electric polarities of the two electric electrodes being opposite, electric field lines emitting from one electric electrode to another electric electrode;
an electric measurement meter connected to the two electric electrodes by using two conductive wires; one end of each conductive wire fixed to a respective electric electrode, and the other end of the conductive wire being connected to the electric measurement meter; the electric measurement meter serving to measure the electric properties between the two electric electrodes;
wherein the electric property is mainly affected by the metal impurities between the two electric electrodes; the electric properties are such as resistance, reactance, capacitance, inductance, voltage, current, frequency, etc., the metal impurities is such as iron powders or particles, or powders or particles of other metal, etc. within a fuel tank; and
wherein in use, the frame with the two electric electrodes is placed into a fuel tank and is sunk into the bottom of the fuel tank; and the electric force of the two electric electrodes will attract the metal impurities in the fuel tank and the metal impurities are distributed along the electric lines so that the electric measurement meter can measure the electric properties between the two electric electrodes; by the electric property, it can be used to determine the quantity of the metal impurities within the fuel tank so as to determine whether the fuel tank exists too much metal impurities and is needed to be cleaned.

2. The electric meter for measuring metal impurities in a fuel tank as claimed in claim 1, wherein the frame is a fuel tank measurement ball which is non-magnetic and the magnetic lines can pass therethrough; and
the two electric electrodes are two magnetic electrodes which are located at a platform surface of the measurement ball; polarities of the two magnetic electrodes are opposite; magnetic lines emitting from one magnetic electrode to another magnetic electrode so that magnetic material will distribute along the magnetic lines.

3. The electric meter for measuring metal impurities in a fuel tank as claimed in claim 2, wherein the platform surface are formed with two recesses for locating the two magnetic electrodes.

4. The electric meter for measuring metal impurities in a fuel tank as claimed in claim 2, wherein an opened channel is formed in the measurement ball with two openings which are in the platform surface; a non-magnetic bar with two magnetic electrodes at two ends thereof is received in the channel with the two magnetic electrodes being exposed in the two openings.

5. The electric meter for measuring metal impurities in a fuel tank as claimed in claim 2, wherein magnetic force between the two magnetic electrodes is adjusted so that the magnetic force is formed only in a nearby of the measurement ball; and thereby, the frame with the two electric electrodes placed within the fuel tank is not adhered to a wall of the fuel tank.

6. The electric meter for measuring metal impurities in a fuel tank as claimed in claim 2, wherein an outer surface of the measurement ball is non-flat and concave so as to have larger area for attracting metal impurities.

7. The electric meter for measuring metal impurities in a fuel tank as claimed in claim 2, wherein the electric measurement meter is used to measure resistance and reactance.

8. The electric meter for measuring metal impurities in a fuel tank as claimed in claim 1, wherein the frame is an enclosure which is non-magnetic and the magnetic lines can pass therethrough; an outer surface of the enclosure is concave so as to have a larger area for attracting metal impurities;
the two electric electrodes are also two magnetic electrodes which are enclosed and fixed within the enclosure;
one end of each of the conductive wires is connected to the respective one of the two magnetic electrodes and the another end thereof is connected to one end of the electric measurement meter so as to measure the electric properties;
the enclosure is two sheet bodies; each sheet body has a recess; the two magnetic electrodes are placed in the respective recesses;
wherein in assembly, the two sheet bodies are combined together; and material to combine the sheet bodies is not conductive so as to avoid the two sheet bodies from conduction to each other; because the two magnetic electrodes are made of non-magnetic material and magnetic lines can passed therethrough so that magnetic lines of the two magnetic electrodes can pass through the sheet bodies to attract the metal impurities.

9. The electric meter for measuring metal impurities in a fuel tank as claimed in claim 8, wherein in assembly, the two sheet bodies are combined together by screwing, using stud, gluing, buckling.
